(19) United States
(12) Patent Application Publication
(10) Pub. No.: US 2015/0297412 A1
(43) Pub. Date: Oct. 22, 2015

(54) Diaper Wound Isolation Method used to treat excoriated diaper dermatitis

(71) Applicant: Joy Cameron-Lawson, Hialeah, FL (US)

(72) Inventor: Joy Cameron-Lawson, Hialeah, FL (US)

(21) Appl. No.: 14/691,497

(22) Filed: Apr. 20, 2015

Related U.S. Application Data
(60) Provisional application No. 61/981,605, filed on Apr. 18, 2014.
(60) Provisional application No. 62/007,744, filed on Jun. 4, 2014.
(60) Provisional application No. 62/150,168, filed on Apr. 20, 2015.

(51) Int. Cl. A61F 13/02; A61F 13/00

(52) U.S. Cl. CPC A61F 13/023; A61F 13/00072; A61F 13/00021; A61F 13/00034; A61F 2013/0017; A61F 2013/00182; A61F 2013/00187

(57) ABSTRACT

A Method to treat excoriated diaper area or diaper wounds is disclosed including utilizing a porous wound covering and placement of at least one medicament on a distal surface of said covering, whereby said method results in shorter healing time.

### INDEX TO RELATED APPLICATIONS

This application is a non-provisional of, and claims benefit to each of U.S. Provisional Patent No. 61/981,605 filed Apr. 18, 2014; 62/007,744, filed Jun. 14, 2014, and 62/150,168, filed Apr. 20, 2015, the disclosures of which are incorporated herein by reference in their entirety.

### BACKGROUND OF THE INVENTION

Diaper Dermatitis (DD) is an inflammatory condition that affects Neonatal Intensive Care Unit (NICU) patients worldwide. Barrier products in the form of creams and pastes are mainstay treatment. Once paste or cream is applied, it is stuck to the diaper offering less protection of the skin from excrements. Babies are developmentally incontinent; the zone exposed to excrements must be protected with a more durable product to protect present excoriation and prevents new excoriation of skin in this zone. This product changes current practice. It provides a new approach to the management of an old problem.

### SUMMARY OF THE INVENTION

Presently there is no set method to care for excoriated diaper dermatitis. Creams and pastes are the mainstay treatment for this problem because these wounds began as rashes. The Diaper Wound Isolation Method (DWIM) of the present invention was developed with the idea of changing the management of excoriated diaper dermatitis by redefining this skin condition from that of a rash to that of a wound. This redefinition of the rash to a wound provided a new opportunity for a different approach; hence, as a wound the availability of products and the method to manage changes.

### BRIEF DESCRIPTION OF THE SEVERAL VIEWS OF THE DRAWINGS

FIG. 1 is a top perspective view demonstrating placement of gauze on infant according to one embodiment the present invention.

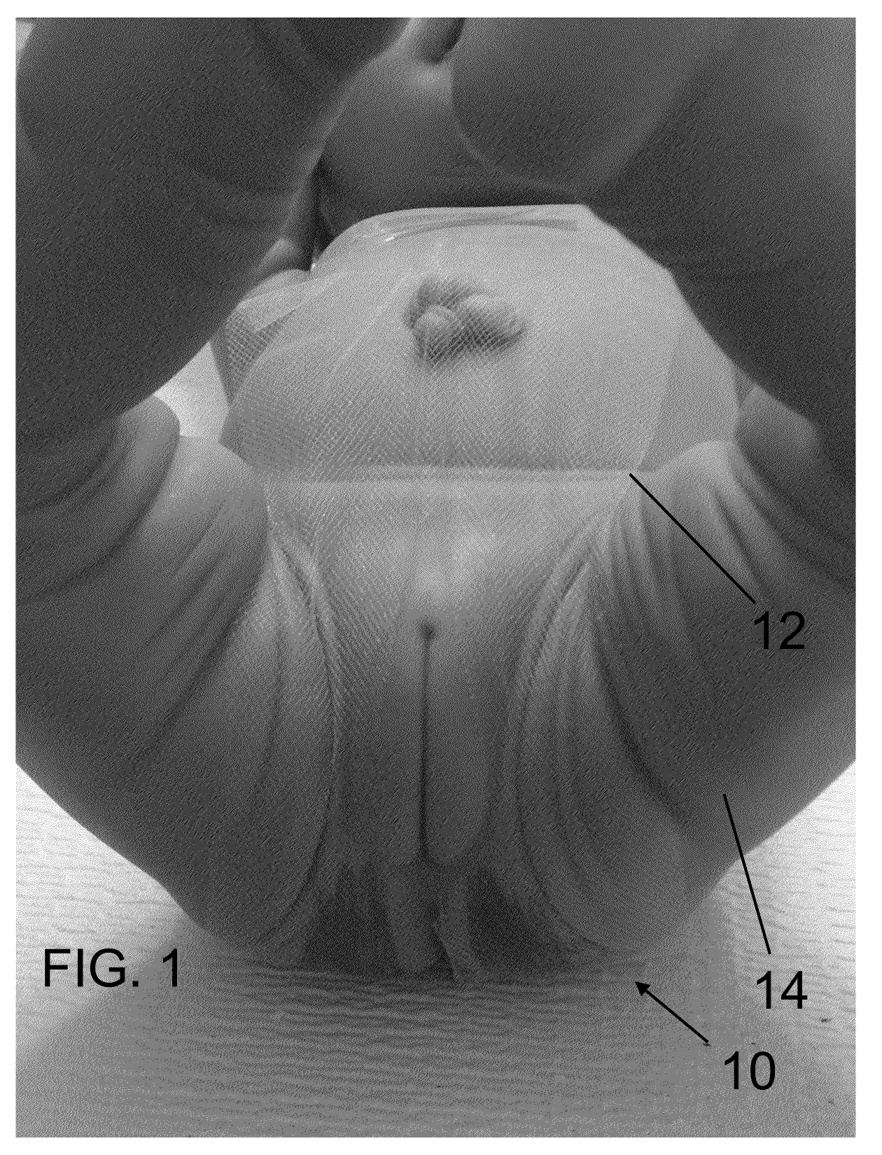

FIG. 2 is a rear perspective view demonstrating placement of gauze on an infinite point to one embodiment of the present invention.

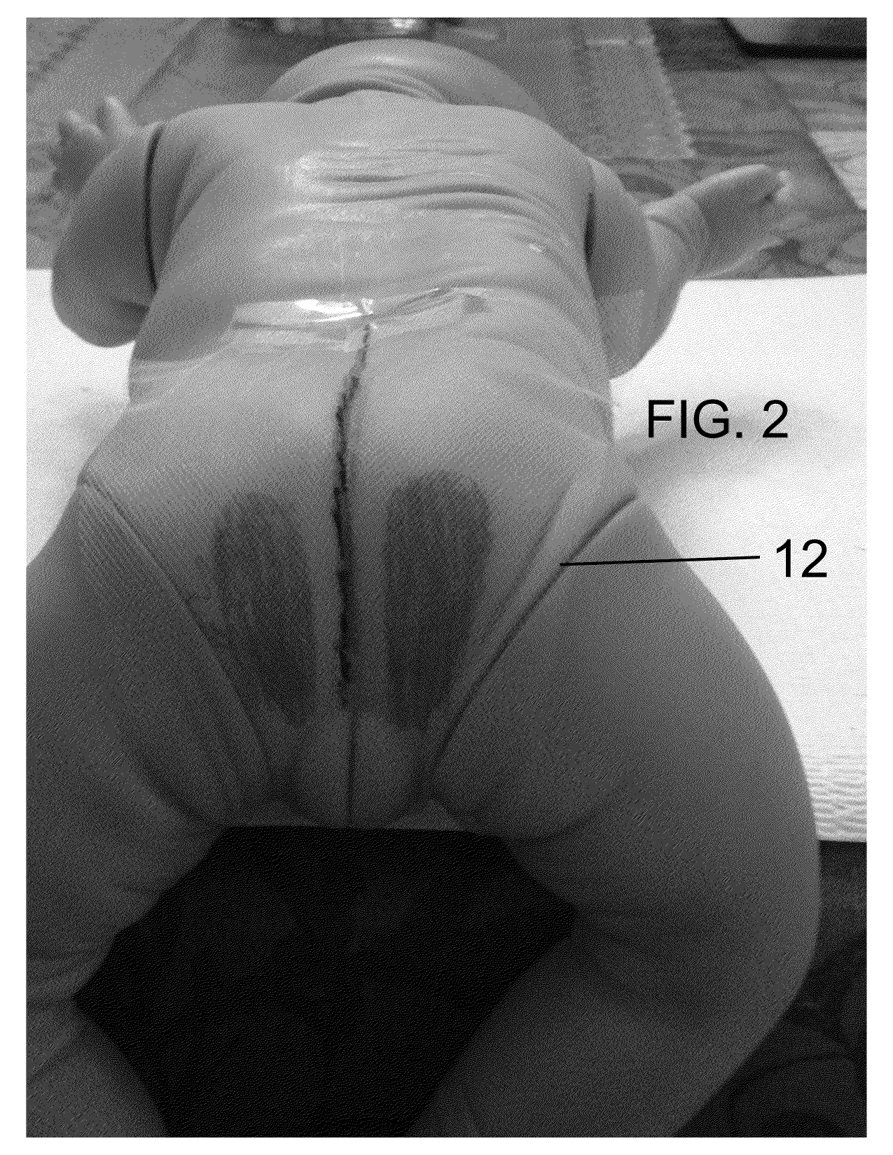

FIG. 3 is a rear perspective view demonstrating placement of at least one medicament on the distal side of the gauze according to one embodiment of the present invention.

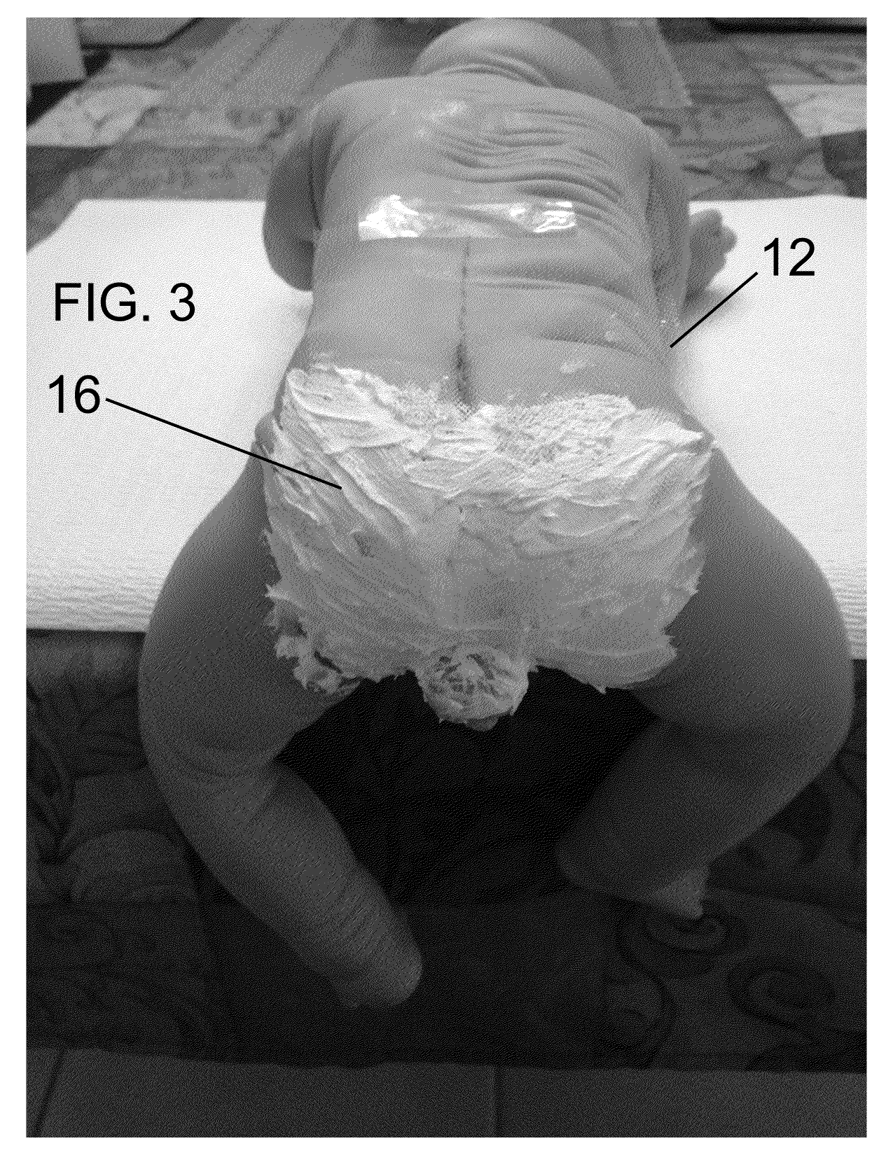

FIG. 4 is a rear perspective view with the gauze or partially removed and showing medicaments that has passed through the previously placed gauze.

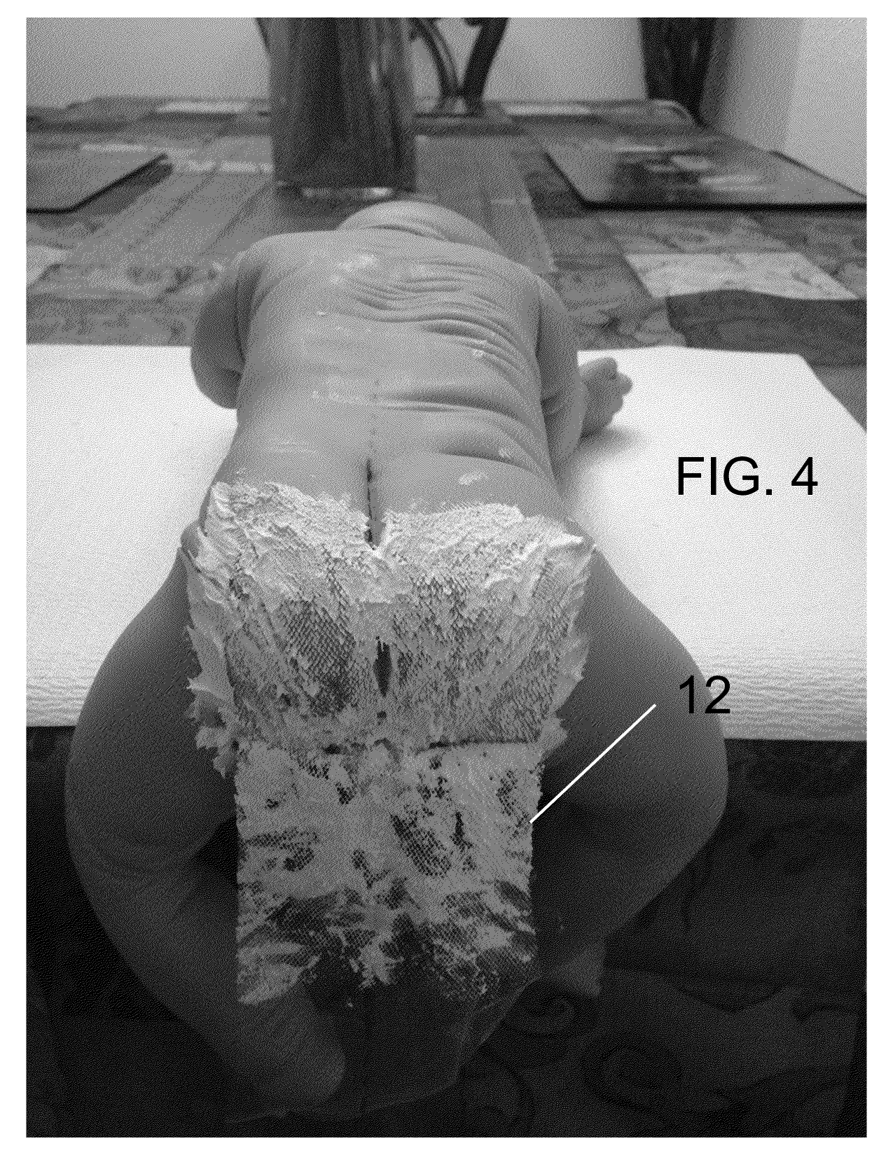

FIG. 5 is a front perspective view with the gauze or partially removed and showing medicaments that has passed through the previously placed gauze.

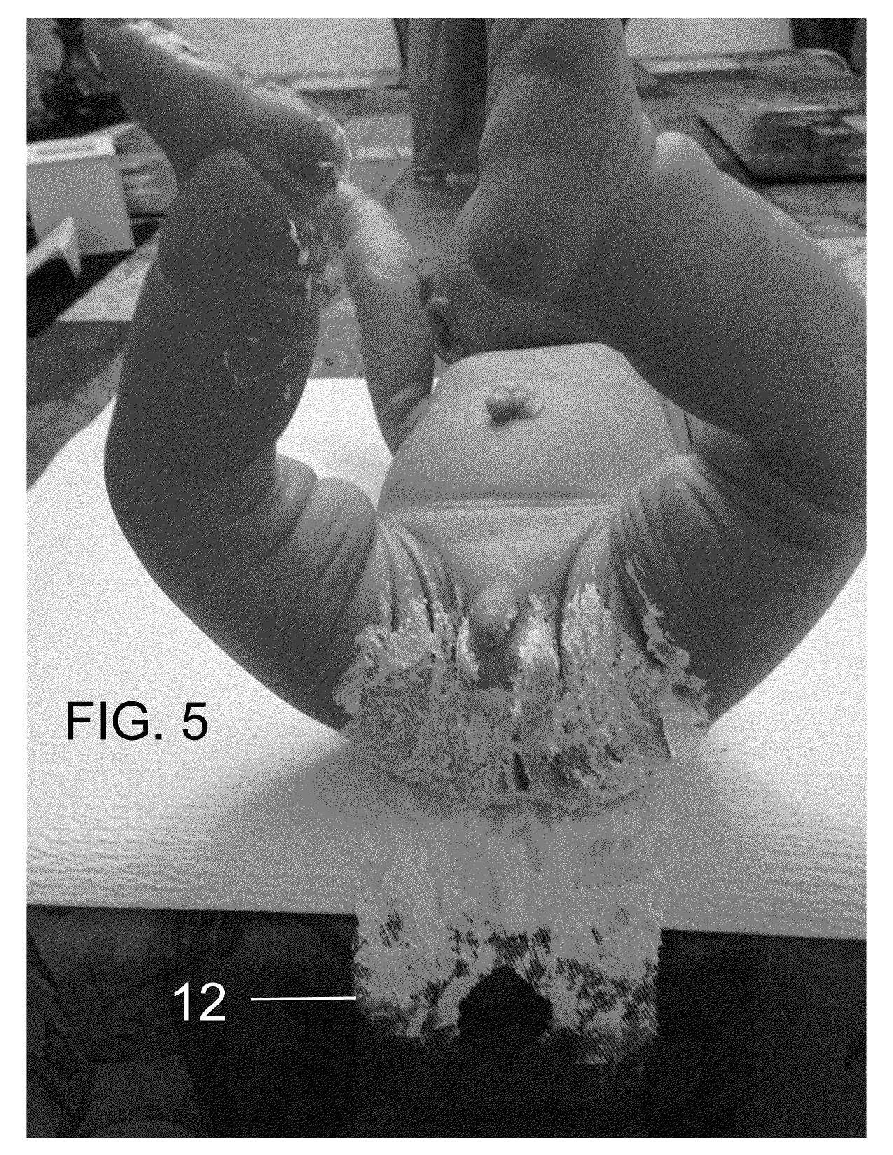

FIG. 6 is a front perspective view demonstrative of placement of a secondary material over the positioned gauze as demonstrated in FIGS. 1 through 5.

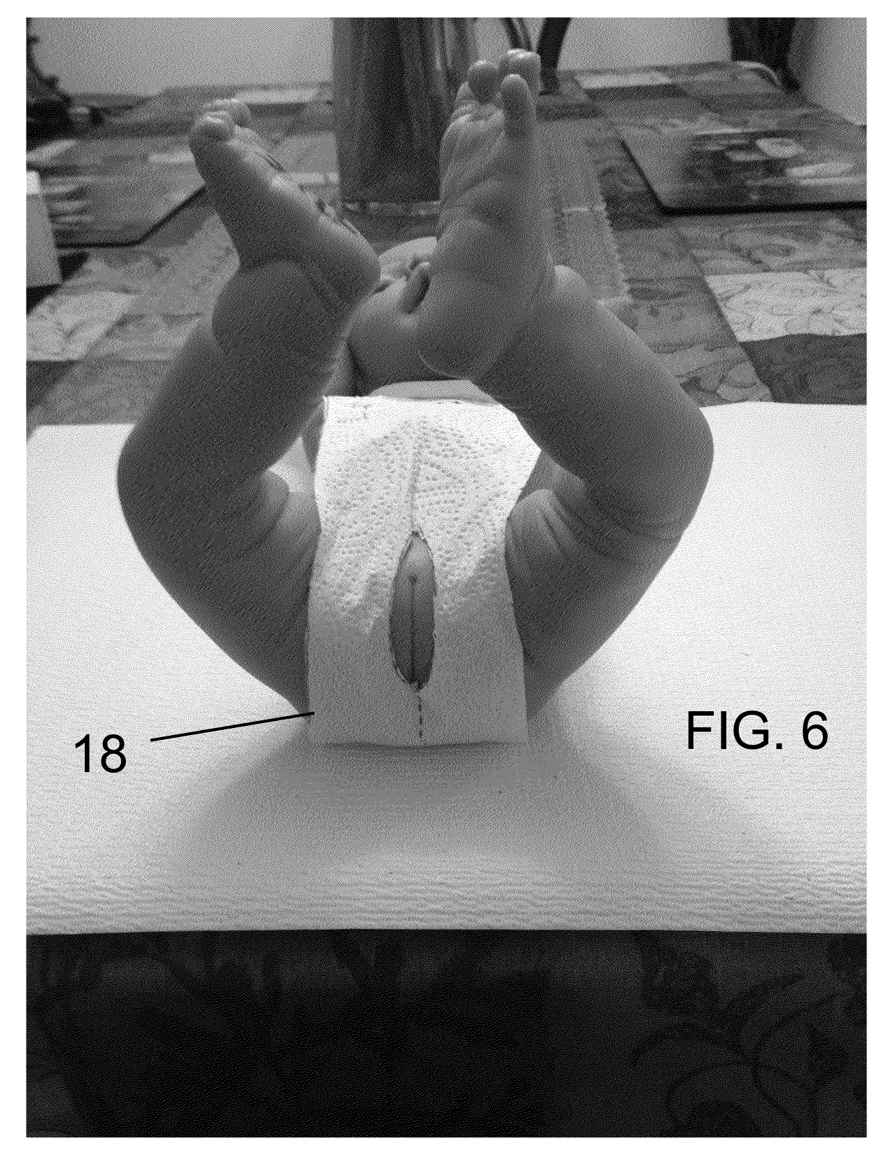

FIG. 7 is a rear perspective view demonstrative of placement of a secondary material over the positioned gauze as demonstrated in FIGS. 1 through 5.

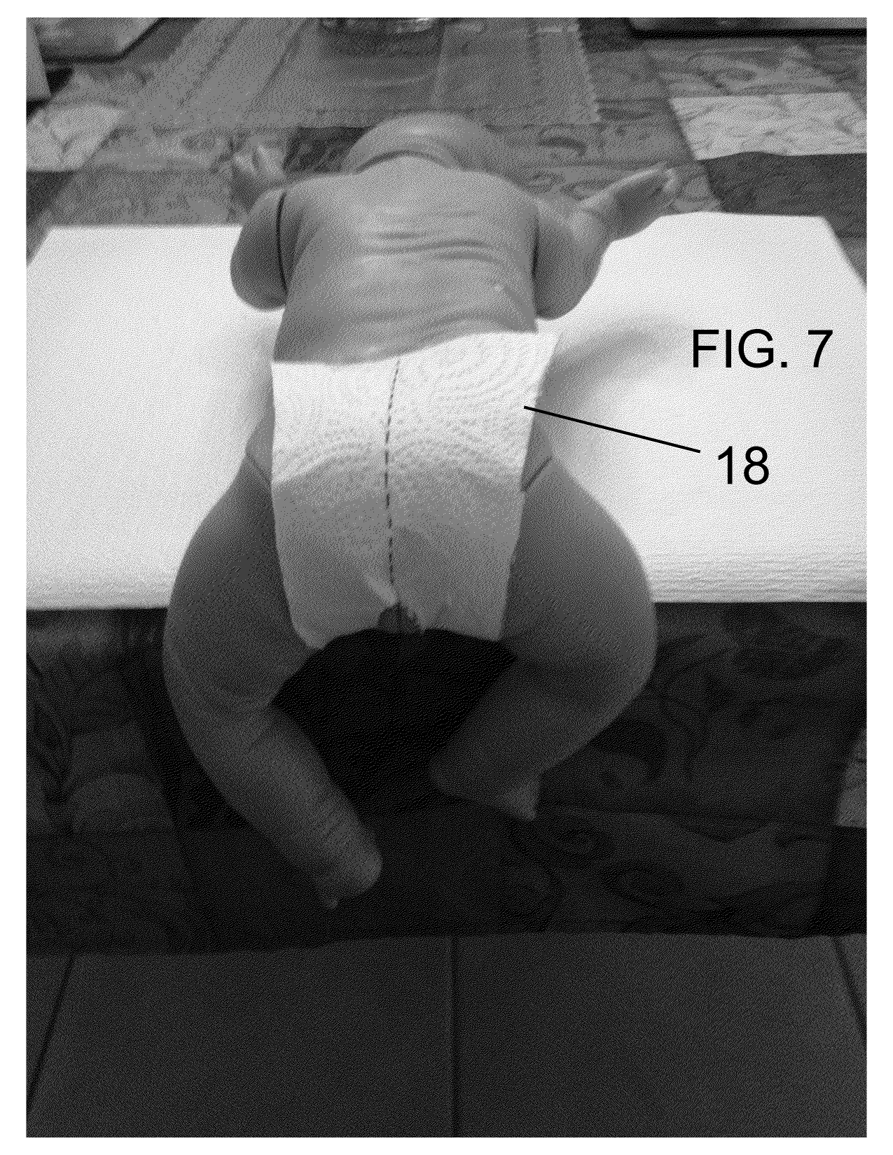

### DETAILED DESCRIPTION OF THE PREFERRED EMBODIMENT

The present invention is a system and method for treating and ameliorating excoriated diaper dermatitis (EDD). This condition is significantly more severe than the conventionally observed diaper rash. Patients with this condition can experience severe pain, discomfort, bleeding, and possibly permanent skin damage. The system and method of the present invention goes against conventional wisdom that requires application of a medicament directly onto the affected skin area.

The present invention has discovered that a treatment regimen that includes a porous barrier directly upon the skin and application of at least one medicament onto the distal surface of the porous barrier provides significantly improved efficacy in the treatment of EDD.

System 10 includes a porous barrier 12. Suitable porous barriers include particular gauze and gauze like materials as described herein that are formed with materials that do not ordinarily irritate skin surfaces. Porous barrier 12 is placed upon patient 14 as demonstrated by the figures. Placement is generally at or around the waist line on the front of the patient is extending past the genital region, under the trunk and between the legs, and terminating above the buttocks at or near the waistline on the back of the patient.

At least one medicament 16 is placed on the distal or outer surface of porous material 12. It is contemplated that by placing the medicament on porous material 12 and not placing it directly upon the skin of the patient, a microenvironment is created whereby there are certain areas in which medicament is not present on the skin of the sort of a patient. This microenvironment allows for increased healing of EDD.

A secondary cover 18 is placed over porous material 12 whereby secondary cover is constructed and arranged to be securely fastened and to hold porous material 12 in a substantially fixed position.

This product is like a skin. It will create a micro-environment around the excoriated area and when used in accordance with the DWIM it will not only protect or isolate the diaper wound from continued exposure to urine and stool; but also, from trauma relating to commercial products and harmful caregiving practices of wiping, dabbing, and scrubbing. This product is designed to work in conjunction with a protective barrier paste. It is designed for diaper wounds or wounds in the diaper area that are superficial and/or partial thickness in nature that began as a rash.

This invention is an improvement on what currently exists. Currently there are only creams and paste being used to manage diaper excoriations in babies. Also, caregivers using those methods vary in their practices. This dressing is designed to be part of a method—The Diaper wound isolation method (DWIM) that standardizes practice as well as provide an infrastructure for alternate medical management as in cases complicated with mycosis. This dressing in the short term can be impregnated to be used for similar type diaper excoriation across the lifespan in human, and in the long term, it can be designed for safe use under phototherapy for hospitalized newborns with excoriated DD.

CHARACTERISTICS OF THE DIAPER WOUND DRESSING:

1. This is a dressing that comes in male and female versions.
2. Shaped to match the convexity of the body, like a sanitary pad, but wider.
3. It is ultra-thin and perforated like a mesh to make the dressing breathable.
4. Male dressing has an almond shaped opening for the genitalia.
5. Female dressing has an oval or teardrop shaped opening for the genitalia.
6. Dressing comes with potential for perforation to expose the anus once dressing is applied.
7. Dressings will be supplied individually packaged in a peel and used fashion similar to disposable gloves/sanitary pads.
8. Dressing has the potential to be impregnated with medication.
9. Dressing may have an optional adherent agent to both extreme ends. Adherent agent is intended to be non-stripping or non-irritating and serves to keep dressing in place.
10. Dressing is designed to be used in conjunction with a barrier paste.
11. Dressing is designed to be changed every diaper change without the worry of disruption of the epithelialization process.
12. Dressing is designed to be irrigated in place if needed.
13. Dressing is part of the Diaper Wound Isolation Method (DWI Method).

### Dressing Design

This dressing will be made from products that are safe for the preterm and/or term newborn's skin. Dressing will be anatomically shaped for male and female. Male dressing will be almond shaped and female will be tear drop shaped.

Dressing will have a gauze infrastructure and thread formation in a matrix or lattice design. Gauze will be cellulose/silicone impregnated. Dressing is designed to be wider than a sanitary pad, it will be breathable, non-occlusive, non-stripping, non-adherent, designed to tolerate irrigation, and has the potential to be impregnated with medication.

### How Dressing Works

This product is constructed and used with a protective barrier paste that is applied generously once the dressing is in place. The dressing can be left in place undisturbed preferably, but can be changed without interrupting the healing process. The area will be irrigated with each diaper change with normal saline solution. A new dressing and coat of barrier paste will be reapplied. No commercial wipes are allowed. The act of wiping, dabbing, drying is not allowed.

### Summary of Use of Dressing

1. THE DRESSING BY DESIGN IS ANATOMICAL BREATHABLE, NON-OCCLUSIVE, AND NON-ADHERENT AND SERVES TO ISOLATE THE WOUND.
2. THE DRESSING'S POSITION IS FORTIFIED BY THE APPLICATION OF A PROTECTIVE BARRIER PASTE.
3. THE DRESSING IS FIRST PART OF THE DIAPER WOUND MICRO-ENVIRONMENT WHICH MUST BE PRESERVED TO PROTECT THE RE-EPITHELIALIZATION PROCESS. THEREFORE, NO COMMERCIAL WIPES, ACT OF WIPING, DABBING, DRYING IS ALLOWED POST IRRIGATION BETWEEN DIAPER CHANGES.

### Experimental Data and Results

Cases were diagnosed with excoriated diaper dermatitis by Medical Doctor (MD) and were referred post treatment with other conventional methods.

This method, referred to as the diaper wound isolation method (DWI method) creates a micro-environment in which the diaper wound is first isolated with a non-adherent silicone dressing which is then supported with a generous application of a high concentration petrolatum/zinc paste. The diaper wound is then irrigated between diaper changes with normal saline to decrease the bio-burden of the wound—the bio burden being the excrement from stool and urine. Caretakers are as different as their practices and may delay or increased the healing time of these wounds. Consequently, no commercial wipes, act of wiping, dabbing or drying is allowed between diaper changes because the high petrolatum content of the protective barrier paste repels the liquid. Also, if the protective barrier was applied generously, excrement will make contact first with this layer, while the wound remains protected by the silicone dressing. A new dressing is applied at each diaper change. Protective barrier paste is then re-applied generously. This process is repeated at each diaper change.

This process is continued until wound is fully healed. Because these wounds are primarily superficial and affect primarily the epidermal layer of the skin, they can be classified as partial thickness injuries. Most partial thickness wounds, when treated appropriately as per evidence will heal within seven (7) days of wound treatment. Consequently, seven days was used as the possible duration using the DWIM. The cases looked at so far showed consistent progress either being fully healed or almost healed at time of discharge. Some cases were discharge home midway while on treatment. All products needed, except for anatomical dressing, are marketed individually; but can be assembled in a kit for single personal use. A generic silicone dressing was used in strip form in the clinical quality improvement project using the DWIM. The anatomical dressing is discussed in another provisional patent.

### Case Study 1

A 35 week gestational age Hispanic male neonate with a birth weight of 1.735 kilograms was delivered via emergent cesarean section(C/S). He was admitted to the NICU for respiratory distress, sepsis, possible genetic malformation relating to trisomy 18, feeding intolerance, and gastro-esophageal regurgitation disorder (GERD). He had frequent stools that led to the excoriation of the buttocks and base of his scrotum. The DD was treated by routine NICU regimen that included nystatin cream and exposure to air/oxygen for 20 days without improvement. The DWI method was initiated at day 20 of failed routine DD treatment for a total of 7 days. After 7 days of the DWI method, the diaper wound was significantly improved.

### Case Study 2

A 31 weeks gestational age African American female neonate with a birth weight of 1.485 kilograms delivered via emergent c/s. She was admitted to the NICU for tachypnea of the newborn versus pneumonia, possible sepsis due to prolonged rupture of membrane (PROM), and hyperbilirubinemia. She developed DD 1 day after admission. The infant's DD was treated by routine NICU diaper regimen with blow-by oxygen, magic paste made from questran, and nystatin cream for 7 days. The DD had become larger, deeper and more inflamed and was classified as a wound. The routine method was discontinued and The DWI method was initiated. After 4 days using the DWI method the wound was mostly re-epithelialized. Pt was discharged home on day four of treatment prior to the completion of treatment.

### Case Study 3

A 36 weeks gestational age African American male with a birth weight 4.485 kilograms. He was delivered via repeat cesarean section. He was admitted to the NICU for infant of a diabetic mom (IDM), transient tachypnea of the newborn (TTN) versus pneumonia, possible sepsis due to unknown maternal group beta streptococcus (GBS) status, and cardiomegaly. He developed a DD that was treated with magic paste at first and then vitamin A & D ointment. The magic paste used consisted of ilex paste, aquaphor, and aluminum hydroxide and it was used for two (2) days. Vitamin A & D ointment was also used for two (2) days. The DWI method was initiated after four days of failed DD treatment. The wound was completely re-epithelialized by four (4) days of treatment.

### Case Study 4

This is a 28 weeks Caucasian female Twin A, who was 0.715 kilogram at birth. She was delivered via emergent c/s. She was admitted for prematurity, respiratory distress syndrome (RDS), neonatal sepsis, hyperbilirubinemia, hyponatremia, and DD. She had frequent stools which led to the excoriation of her buttocks. She received zinc oxide for 20 days. She was then changed to magic paste which was a formulation of zinc, nystatin, cholestyramine and treated for eight days additional. Clinical condition continues to worsen and the DWI method was initiated after 28 days of failed DD management. The wound was completely re-epithelialized by seven (7) days of initiation of treatment.

### Case Study 5

This is a 28 weeks gestational age Caucasian female, Twin B, who was 0.590 kilogram at birth. She was delivered via emergent c/s. While hospitalized, she was treated for prematurity, RDS, neonatal sepsis, hyperbilirubinemia. Pt had a DD which was treated with Bacitracin ointment and then Vitamin A &D ointment for a total of 13 days. The DWI method was initiated after 13 days of failed DD management. The wound was completely re-epithelialized by seven (7) days of treatment.

### Case Study 6

This is a 32 weeks gestational age Hispanic male who was 1.050 kilograms at birth. He was delivered via emergent c/s. While hospitalized, he was treated for respiratory distress syndrome, anemia, thrombocytopenia, neonatal sepsis, congenital cytomegalus virus (CMV), CMV chorioretinitis, hyperbilirubinemia, microcephaly, severe ventriculomegaly, hypoglycemia, and DD. Pt had a DD that was being treated with nystatin cream. DD appeared significantly worse and the treatment was changed to the DWI method, after one day of treatment. The wound was completely re-epithelialized by six (6) days of treatment.

### Case Study 7

This is a 25 weeks gestational age African American female who was 0.815 kilogram at birth. She was delivered via emergent cesarean section. She was admitted to the NICU for respiratory distress, complete heartblock due to maternal systemic lupus erythematous (SLE), hypotension, neonatal onset of sepsis. She developed a DD which was treated with Vitamin A & D ointment and Magic paste (cholestyramine and zinc oxide) for a total of nine (9) days. The DWI method was initiated on the tenth (10) day of failed DD management. The wound was completely re-epithelialized by seven (7) days of treatment.

### Case Study 8

This is a 31 weeks gestational age Hispanic American male who was 1.320 kilogram at birth. He was delivered via emergent cesarean section due to severe Pre-Eclampsia. He was admitted to the NICU for respiratory distress, hypermagnesemia & feeding intolerance. He developed a DD which was treated with Zinc oxide initially for 2 days, and then Magic paste (Aquaphor and Nystatin) for a total of thirteen (13) days. The DWI method was initiated on the thirteenth (13) day of failed DD management. The wound was completely re-epithelialized by seven (7) days of treatment.

Table 1 sets forth degrees quantifying severity of the patient condition.

TABLE 1
DIAPER WOUND EVALUATION TOOL

| Score | Degree | Definition |
|---|---|---|
| 0 | None | Skin is clear(may have some very slight dryness |
| | | and/or a single papule but no erythema. |
| 0.5 | Slight | Faint to definite pink in a very small area(<2%); |
| | | may also have a single papule and/or slight dryness. |
| 1.0 | Mild | Faint to definite pink in a small area(2%-10%) or |
| | | definite redness in a very small area(<2%) and/or |
| | | scattered papules and/or slight dryness/scaling. |
| 1.5 | Mild/ | Faint to definite pink in a larger area(10%) or |
| | Moderate | definite redness in a small area(2%-10%) or very |
| | | intense redness in a very small area(<2%) and/or |
| | | scattered papules(<10% area) and/or moderate |
| | | dryness/scaling. |
| 2.0 | Moderate | Definite redness in a larger area (10%-50%) or very |
| | | intense redness in a very small area(<2%) and/or |
| | | single to several areas of papules(10%-50%) with five |
| | | or fewer pustules, may have slight desquamation or |
| | | edema. |
| 2.5 | Moderate/ | Definite redness in a very large area(>50%) or very |
| | Severe | intense redness in a small area (2%-10%) without |
| | | edema and/or larger areas(>50%) of multiple papules |
| | | and/or pustules; may have moderate desquamation |
| | | and/or edema. |
| 3.0 | Severe | Very intense redness in a larger area (>10%) and/or |
| | | severe desquamation, severe edema, erosion and |
| | | ulceration; may have large areas of confluent papules |
| | | or numerous pustules/vesicles. |

TABLE 2
CASE STUDIES DATA

| CASES | DAY 1 | DAY 3-4 | DAY 7 |
|---|---|---|---|
| 1 | 3 | 1.5 | 0.5 |
| 2 | 3 | 0.5 | Pt discharged home |
| 3 | 3 | 0.5 | Pt discharged home |
| 4 | 2 | 1.0 | 0 |
| 5 | 2 | 0.5 | 0 |
| 6 | 3 | 0.5 | 0 |
| 7 | 2.5 | 1.0 | 0 |
| 8 | 3.0 | 1.0 | 0 |

Table 2 is a table of values quantifying patient condition and progress.

### Data Summary

The evaluation of cases presented shows that regardless of the clinical course, gestational age, and other clinical factors affecting these patients, there is consistency in the progression of healing using the DWI method. Re-epithelialization of the wound is evident from the onset of treatment. The healing time of diaper wounds is decreased using the DWI method. The DWI method was effective, although it required a few more steps in the management of these patients.

### The Version of the Method or Invention Discussed Here Involves:

1. A non-adherent, breathable silicone dressing

### 2. A Protective Barrier Paste

3. Normal Saline solution
4. A 30-35 mls syringe designed with a 19 gauge catheter tip.
5. Barrier Field (optional)

### Relationship Between the Components:

The non-adherent, perforated silicone dressing will be applied to the cleansed wound in the diaper area. This isolates the wound, but allows it to breathe and be exposed to the medicinal properties of the paste that will be applied. Step 2. Skin protective barrier paste is applied to support the micro-environment of the wound created by the dressing. When the baby stools/urinates the excrement will get to the protective paste first. At the time of the diaper change, the dressing is removed and site is irrigated. Step 3. The catheter tipped syringe is used to collect the normal saline used in the irrigation. Syringe is chosen because it adds pressure to clean site vs. the traumatic exposure that comes from wiping or cleaning done by caretakers. Wiping, dabbing, scrubbing, and using commercial products will disturb the re-epithelialization of the wound and delay healing. The normal saline is chosen because of its non-pyrogenic properties.

This is the isolation of diaper wounds by the creation of a micro-environment using a breathable, non-occlusive, non-adherent dressing; then, supporting this area with a protective barrier paste. Diaper wound site is irrigated at each diaper change. No commercial wipes, act of wiping, dabbing or scrubbing at site during diaper changes. New dressing is applied and protective barrier paste is applied over the dressing generously. Patient (Pt) is allowed to be bathed as usual, but extra care is taken to cleanse wound site during that time. It is recommended to apply a small amount of soap in hand and gently caress wound site during bathes. This method is repeated throughout diaper changes for Seven (7) days.

### How to Make the Invention:

This is a method, and products will be assembled in a convenient kit. Kit will contain:
1. A silicone impregnated gauze dressing which is designed in a single peel away ready to use fashion—similar to female sanitary napkins, the only difference it will be anatomically shaped to be male/female, thin, breathable or porous, and non-adherent. The dressing is discussed in another provisional patent.
2. Normal saline,
3. Protective barrier paste
4. A 30 mls syringe with a 19 gauge catheter tip.
5. A re-usable changing pad.

### How to Use the Invention:

1. Cleanse site in the normal fashion of bathing initially.
2. Then irrigate with normal saline.
3. Apply silicone dressing.
4. Apply generous amount of barrier paste.
5. Dress infant as usual.
6. Irrigate with normal saline each diaper change.
7. No wiping, drying, or commercial wipes allowed.
8. Re-apply a generous amount of barrier paste each diaper change.

### Notes

Between diaper changes, there is no need to attempt to dry wound post irrigation because there is a natural repellence to the liquid irrigant due to the high petrolatum content of the Sensi-Care.

Caregivers are instructed to not attempt to clean residual Sensi-Care protective barrier from the wound because of the possibility of re-injuring of the diaper wound.

The 19-gauge catheter tip is likened to a power hose. It allows for the generation of adequate psi (4-15) for the cleansing of the wound site as best as possible.

Provide daily baths as usual, but diaper area should be gently cleansed to keep fresh.

Avoid scrubbing, abrasive materials to wound area. Rationale: Rough cleaning, abrasive clothes will disturb the re-epithelialization process of the wound and delay or worsen wound.

If there is stool stuck to the barrier paste, during bath time simple place a very small portion of soap in your gloved hand (if in hospital) or in the palm of your hand at home and gently caress wound site. The aim of bath during this time is for comfort and hygiene; fine details will be possible once wound is fully healed.

Diaper excoriations are primarily partial thickness wound types. These wounds were evidenced as having a healing time of seven (7) days.

Wounds that extend beyond the healing time of 7 days while using the DWIM as instructed, may be related to other serious pathologies or skin abnormalities. MD should be notified for consults from specialists such as: Dermatologist/Rheumatologist/oncologist, etc.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A method for treating excoriated diaper dermatitis, said method comprising the steps of:
cleaning an infected area of a patient;
providing a properly sized porous gauze;
placing said gauze onto infected area;
placing at least one medicament on top of said gauze;
covering said gauze with a covering constructed and arranged to secure said cause into a substantially fixed position.

2. The method of claim 1 wherein said placing on infected area includes placing on a patient above genitalia and buttocks of a patient.

3. The method of claim 1 whereby said method demonstrates at least 50% improvement in severity of excoriated diaper dermatitis after three days of treatment.

4. The method of claim 1 whereby said method demonstrates at least 90% improvement in severity of excoriated diaper dermatitis after seven days of treatment.

5. The method of claim 1 wherein said providing a properly sized porous gauze includes providing a particular gauze configured for a male patient or a particular gauze configured for a female patient.

6. The method of claim 1 wherein said method is repeated at each diaper change.

7. The method of claim 1 wherein said method includes providing a kit, said kit including: a silicone impregnated gauze dressing configured in a single peel away ready to use fashion said gauze being anatomically shaped for either male or female, wherein said gauze is a thin, breathable or porous, and non-adherent; normal saline; at least one medicament; a syringe; and a A re-usable changing pad.

8. The method of claim 1 wherein said medicament is a protective barrier paste
